(19) United States
(12) Patent Application Publication
(10) Pub. No.: US 2014/0120181 A1
(43) Pub. Date: May 1, 2014

(54) COMPOSITION COMPRISING PHOSPHATIDYLCHOLINE AS AN ACTIVE INGREDIENT FOR ATTENUATING TOXICITY OF ANTICANCER AGENT

(71) Applicant: AMI Pharm Co., Ltd., Seoul (KR)

(72) Inventors: Ki Teak LEE, Seongnam-si (KR); Jong Hyuk LEE, Seoul (KR); Ji Hoon JEONG, Uiwang-si (KR)

(21) Appl. No.: 14/140,025

(22) Filed: Dec. 24, 2013

Related U.S. Application Data
(63) Continuation of application No. PCT/KR2012/004998, filed on Jun. 25, 2012.

(30) Foreign Application Priority Data
Jun. 24, 2011 (KR) 10-2011-0061658

(51) Int. Cl. A61K 31/685; A61K 31/704; A61K 33/24

(52) U.S. Cl. CPC A61K 31/685; A61K 33/24; A61K 31/704
USPC 424/649; 554/80; 514/78

(57) ABSTRACT

The present invention relates to a new use of phosphatidylcholine, and more particularly to a composition for toxicity reduction of an anti-cancer agent, and an anti-cancer adjuvant, comprising phosphatidylcholine as an active ingredient.

### CROSS-REFERENCE TO RELATED APPLICATIONS

This application is a continuation of International Application PCT/KR2012/004998, filed on Jun. 25, 2012, and claims priority from and the benefit of Korean Patent Application No. 10-2011-0061658, filed on Jun. 24, 2011, both of which are incorporated herein by reference in their entireties for all purposes as if fully set forth herein.

### BACKGROUND OF INVENTION

1. Field

The present invention relates to a new use of phosphatidylcholine, and more particularly to a composition for toxicity reduction of an anti-cancer agent, and an anti-cancer adjuvant, comprising phosphatidylcholine as an active ingredient.

2. Discussion of the Background

Cancer is a disease causing the death of about 7,600,000 people through the world annually, which makes up 13% of all deaths. According to Korea Statistics 2009, statistical annual report on cause of death, cancer accounts for 28.3% of all deaths, and is the leading cause of death in the Korean population. Thus, it is required to take national measures for cancer care. As a method for treating cancer, various methods such as an operation, radiation treatment, gene therapy and the like are currently used. One of the most frequently used therapeutic methods is chemotherapy for administering an anti-cancer agent.

Anti-cancer chemotherapy is a whole body therapy, in which mainly through injection or oral administration, an anti-cancer agent is administered, and is spread throughout the whole body through the blood stream. Accordingly, the therapy acts on micrometastases spread throughout the whole body, rather than causing a local effect. Therefore, it frequently causes side-effects in the whole body, and such side-effects are more serious than that in an operation or radiation treatment. By using a difference in drug-sensitivity between normal cells and cancer cells, the chemotherapy allows an anti-cancer agent to selectively act on the cancer cells. However, there is a problem in that most anti-cancer agents cannot distinguish normal cells from cancer cells, thereby showing dose-limiting toxicity.

A representative anti-cancer agent, cisplatin (cis-diammine-dichloroplatinum [II ]), is a chemotherapy agent for treating ovarian cancer, bladder cancer, lung cancer, head and neck cancer, testicular cancer, etc., and has been clinically widely used (Rosenberg B., Cancer, 55: pp. 2303-2315, 1985). Cisplatin is known to generate oxidative reactive species, thereby attacking cancer cells, and to induce DNA inter-intrastrand cross-linking and DNA adduct formation in the cancer cells, thereby showing an anti-cancer effect.

However, when the drug is used in a larger amount than a limited dose during the is therapy process, side-effects such as hearing loss, neurotoxicity, and nephrotoxicity occur (Mollman et al., 1998; Screnci and McKeage, 1999). Also, when cisplatin at a high-concentration is administered, hepatotoxicity is known to be frequently observed (Cerosimo R. J., Ann. Pharm., 27: pp 438-441, 1993; Cavalli F. et al., Cancer Treat. Rep., 62: pp 2125-2126, 1978; Pollera C. F. et al., J. Clin. Oncol., 5: pp 318-319, 1987).

Such side-effects caused by cisplatin are closely related to an increase in lipid peroxidation by oxidative reactive species generated by cisplatin (Matsushima H. et al., J. Lab. Clin. Med., 131: pp 518-526, 1998; Koc A. et al., Mol. Cell. Biochem., 278(1-2): pp 79-84, 2005), inhibition of antioxidant enzyme activity existing in tissues (Sadzuka Y. et al., Biochem. Pharmacol., 43: pp 1873-1875, 1992), exhaustion of glutathione (Zhang J. G. and Lindup W. E., Biochem. Pharmcol., 45: pp 2215-2222, 1993) and destruction of intracellular calcium hemeostasis (Zhang J. G. and Lindup W. E., Toxicology in Vitro, 10: pp 205-209, 1996).

Paclitaxel is a natural cytotoxic material extracted from Taxus brevifolia bark by the National Cancer Institute (NCI) late in the 1960s, which is a mitosis inhibitor inhibiting cell division, and is one of the most currently spotlighted anti-cancer agents actively acting on malignant tumors such as melanoma, breast cancer, and lung cancer. However, it may act on other normal cells in the body as well, thereby causing other diseases. Also, it has been pointed out that the material seriously causes toxicity and side-effects due to its low water-solubility.

Accordingly, in order to reduce side-effects caused by treatment with an anti-cancer agent and to improve therapy efficiency, it is required to develop an inhibitor that can relieve toxicity caused by administration of an anti-cancer agent.

### SUMMARY

Accordingly, the inventors conducted a study on a novel material capable of relieving anti-cancer agent toxicity. As a result, they found that phosphatidylcholine can relieve toxicity of an anti-cancer agent. Then, based on this finding, they completed this invention.

Accordingly, an object of the present invention is to provide a composition for toxicity reduction of an anti-cancer agent comprising phosphatidylcholine as an active ingredient.

Another object of the present invention is to provide an anti-cancer adjuvant comprising phosphatidylcholine as an active ingredient.

Still another object of the present invention is to provide a method for reducing toxicity of an anti-cancer agent comprising administering an effective amount of phosphatidylcholine to a subject in need thereof.

Still another object of the present invention is to provide use of phosphatidylcholine for preparing a composition for toxicity reduction of an anti-cancer agent comprising phosphatidylcholine.

To achieve the above object, the present invention provides a composition for toxicity reduction of an anti-cancer agent comprising phosphatidylcholine as an active ingredient.

To achieve another object, the present invention provides an anti-cancer adjuvant comprising phosphatidylcholine as an active ingredient.

To achieve still another object, the present invention provides a method for reducing toxicity of an anti-cancer agent comprising administering an effective amount of phosphatidylcholine to a subject in need thereof.

To achieve still another object, the present invention provides use of phosphatidylcholine for preparing a composition for toxicity reduction of an anti-cancer agent comprising phosphatidylcholine.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows a test result graph obtained by comparatively measuring reduction effects of BUN (blood urea nitrogen) level by intra-peritoneal injection of the composition of the present invention (Control: a control group administered with a saline solution, PC: a group administered with phosphatidylcholine (PC) 400 mg/kg, CDDP: a group administered with cisplatin (CDDP) 5 mg/kg, PC400: a group administered with CDDP 5 mg/kg and PC 400 mg/kg, PC600: a group administered with CDDP 5 mg/kg and PC 500 mg/kg, and PC800: a group administered with CDDP 5 mg/kg and PC 600 mg/kg).

FIG. 2 shows a test result graph obtained by comparatively measuring reduction effects of blood creatinine level by intra-peritoneal injection of the composition of the present invention (Control: a control group administered with a saline solution, PC: a group administered with phosphatidylcholine (PC) 400 mg/kg, CDDP: a group administered with cisplatin (CDDP) 5 mg/kg, PC400: a group administered with CDDP 5 mg/kg and PC 400 mg/kg, PC600: a group administered with CDDP 5 mg/kg and PC 500 mg/kg, and PC800: a group administered with CDDP 5 mg/kg and PC 600 mg/kg).

FIG. 3 shows a photograph of a result of microscopic observation of morphological changes in kidney tissues (A. a photograph of kidney tissues of a non-treated normal rat, B. a photograph on kidney tissues of a rat injected with cisplatin in a dose of 5 mg/kg, and, C. a photograph on kidney tissues of a rat injected with cisplatin in a dose of 5 mg/kg and phosphatidylcholine in a dose of 600 mg/kg).

FIG. 4A shows a test result graph obtained by comparatively measuring reduction effects of BUN (blood urea nitrogen) level by oral administration of the composition of the present invention (Y axis-concentration of BUN (mg/dl)).

FIG. 4B shows a test result graph obtained by comparatively measuring reduction effects of creatinine level by oral administration of the composition of the present invention (Y axis-concentration of creatine (mg/dl)).

FIG. 5 shows a test result graph obtained by comparatively measuring reduction effects of MDA (malondialdehyde) level in kidney tissue by oral administration of the composition of the present invention (Y axis-content of MDA per 1 g of kidney tissue (μM/g)).

FIG. 6 shows a test result graph obtained by comparatively measuring increasing effects of total GSH (glutathione) concentration in kidney tissue by oral administration of the composition of the present invention (Y axis-content of GSH per 1 mg of protein (nmol/mg)).

FIG. 7A shows a test result graph obtained by comparatively measuring increasing effects of CAT (catalase) activity in kidney tissue by oral administration of the composition of the present invention (Y axis-content of catalase activity per 1 mg of protein (mmoles/min/mg)).

FIG. 7B shows a test result graph obtained by comparatively measuring increasing effects of GPx (glutathione peroxidase) activity in kidney tissue by oral administration of the composition of the present invention (Y axis-content of GPx activity per 1 mg of protein is (Unit/mg)).

FIG. 7C shows a test result graph obtained by comparatively measuring increasing effects of SOD (superoxide dismutase) activity in kidney tissue by oral administration of the composition of the present invention (Y axis-content of SOD activity per 1 mg of protein (mmoles/min/mg)).

The X axes of FIGS. 4 to 7 have the same meaning. (Normal: a control group administered with a saline solution (group 11 of table 4), PC: a group administered with phosphatidylcholine (PC) 600 mg/kg (group 13 of table 4), CDDP: a group administered with cisplatin (CDDP) 6 mg/kg, PC300: a group administered with CDDP 6 mg/kg and PC 300 mg/kg (group 14 of table 4)), PC600: a group administered with CDDP 6 mg/kg and PC 600 mg/kg, and PC1200: a group administered with CDDP 6 mg/kg and PC 1200 mg/kg).

FIG. 8 shows a test result graph of reduction effects of parclitaxel toxicity according to administration dosage of phosphatidylcholine (X axis-A: a group administered with phosphatidylcholine (PC) 0 mg/kg, B: a group administered with phosphatidylcholine (PC) 300 mg/kg, C: a group administered with phosphatidylcholine (PC) 600 mg/kg/Y axis-administration dosage of parclitaxel for LD₅₀ (mg/kg)).

### DETAILED DESCRIPTION OF ILLUSTRATED EMBODIMENTS

The present invention provides a composition for toxicity reduction of an anti-cancer agent, comprising phosphatidylcholine as an active ingredient.

The composition of the present invention for toxicity reduction is characterized in that it comprises phosphatidylcholine as an active ingredient.

Phosphatidylcholine is a phospholipid widely existing in animals, plants, yeast, and fungi, which is also called lecithin, and corresponds to 1,2-diacyl-L-3-glycerylphosphorylcholine. It is a phospholipid for mammal membrane constitution, and mainly exists in brains, nerves, blood corpuscles, yolks or the like. In plants, it is contained in soybeans, sunflower seeds, wheat germ or the like, and is hardly found in bacteria. In general, at the 1-position of glycerol, a saturated fatty acid, and at the 2-position, an unsaturated fatty acid is bound. An acyl group mostly has 12 to 22 carbon atoms (C12 to C22). The phospholipid exists as an amphoteric ion in all pH ranges since its component, choline, has a pK of about 13. Thus, it has surface activity.

Phosphatidylcholine according to the present invention has a basic structure of <Formula 1>.

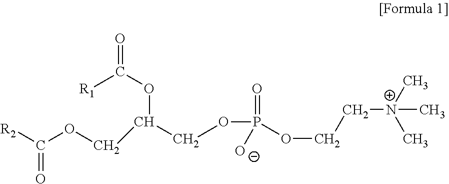

Phosphatidylcholine according to the present invention has a basic structure of <Formula 1> above, wherein R1 may represent C12 to C22 saturated or unsaturated fatty acid, and R2 may represent C12 to C22 saturated or unsaturated fatty acid. Phosphatidylcholine according to the present invention may be a single compound, or a mixture of different compounds having various numbers of carbon atoms of acyl groups of R1 and R2.

Preferably, Phosphatidylcholine according to the present invention may be a mixture comprising the compound having a structure of <Formula 2>, in a ratio of 94.0 wt % or more.

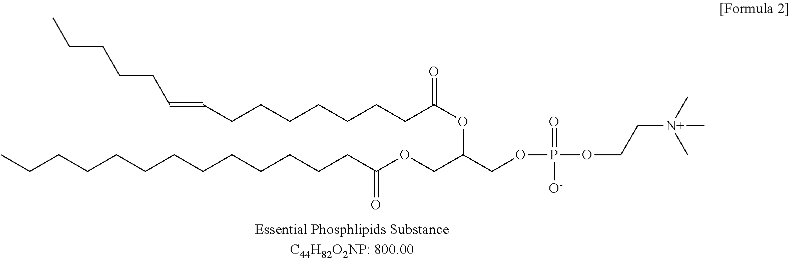 [SMILES: CCCCC/C=C/CCCCCCCC(=O)OC(COC(=O)CCCCCCCCCCCCC)COP(=O)([O-])OCC[N+](C)(C)C]

Phosphatidylcholine according to the present invention may be extracted for use, from any one selected from the group consisting various kinds of animals, or plants, for example, soybeans, sunflower seeds, wheat germ and yolks. Phosphatidylcholine according to the present invention may be preferably separated from soybeans or eggs. Otherwise, Phosphatidylcholine according to the present invention may be bought as a commercially available product.

In one Example of the present invention, 10 kg of soybeans (scientific name: Glycine max (L.) Merill) were washed, peeled and grounded, and then at room temperature, extracted with ethanol (E.P) for 40 min to obtain phosphatidylcholine. Additionally, the obtained extract was filtered to remove proteins and carbohydrates, and then was vacuum evaporated at 40° C. Then, the concentrated extract was degummed and dried to remove moisture, and added with acetone. The acetone layer was separated and the residue was extracted with ethanol at 35° C. or less for 60 min. The extract was purified with silica gel chromatography and aluminum is oxide chromatography so as to provide phosphatidylcholine (essential phospholipids substance) 4 g (yield: 0.04%).

An anti-cancer agent is a general term for drugs that show cytotoxicity or growth inhibiting effects (cytostatic effect) on cancer cells by acting on various kinds of metabolic pathways of the cancer cells. Anti-cancer agents which have been developed until now are divided into antimetabolite, herbal alkaloid, topoisomerase inhibitor, alkylating agent, anti-cancer antibiotics, hormone drug, and other drugs according to its action mechanism and chemical structure.

The anti-cancer agent of the present invention, for example, may be oxaliplatin, imatinib, docetaxel, pemetrexed, gefitinib, tegafur, capecitabine, elotidib, doxifluridine, paclitaxel, interferon alpha, gemcitabine, fludarabine, irinotecan, carboplatin, cisplatin, taxotere, doxorubicin, epirubicin, 5-fluorouracil, UFT, tamoxifen, goserelin, hereceptin, anti-CD20 antibody, leuprolide (lupron) or flutamide, preferably cisplatin or paclitaxel.

Cisplatin (cis-dichlorodiammineplatinum) is a representative anti-cancer agent, which is clinically widely used as a chemotherapy agent for treating ovarian cancer, bladder cancer, lung cancer, head and neck cancer, testicular cancer, etc. Cisplatin is known to generate oxidative reactive species, thereby attacking cancer cells, and to induce DNA inter/intrastrand cross-linking and DNA adduct formation in the cancer cells, thereby showing an anti-cancer effect. However, when the drug is used in a larger amount than a limited dose during the therapy process, side-effects such as hearing loss, neurotoxicity, and nephrotoxicity occur. Also, when cisplatin at a high-concentration is administered, hepatotoxicity is known to be frequently observed.

Paclitaxcel has an action mechanism in which it binds to microtubules participating in carrying of various substances such as chromosomes, and maintaining of cytoskeleton, within cancer cells, and prevents chromosomes of the cancer cells from moving, thereby leading the cancer cells to death. However, it may act on other normal cells in the body as well, thereby causing other diseases. Also, it has been pointed out that, the material seriously causes toxicity and side-effects due to its low water-solubility

Anti-cancer agents have various intracellular targets according to their kinds. They block DNA replication, transcription, and translation processes of cells or inhibit protein action that is important in cell survival. Then, such an effect on an intracellular target leads the cells to death through necrosis or apoptosis. However, such a metabolic pathway on which the anti-cancer agents act is not specific to only cancer cells, but is the same to normal cells as well. Thus, when the anti-cancer agents are administered, damage to normal tissues, that is, toxicity, is unavoidable.

In the present invention, toxicity of an anti-cancer agent may be nephrotoxicity, hepatotoxicity, neurotoxicity, blood toxicity, gastrointestinal toxicity, or pulmonary toxicity, preferably nephrotoxicity, blood toxicity, or neurotoxicity.

There was reported that paclitaxel has side-effects of leucopenia and neurotoxicity (S. M. Lichtman et. al., Ann Oncol; 23 (3): 632-638, 2012).

In the present invention, an anti-cancer agent may be of any type as long as it is an anti-cancer agent having a cancer inhibiting and treating effect. Also, there is no particular limitation in the kind of cancer, but the cancer may be preferably any one selected from the group consisting testicular cancer, bladder cancer, prostate cancer, ovarian cancer, breast cancer, colorectal cancer, head and neck cancer, lung cancer, esophageal cancer, stomach cancer and uterine cervical cancer.

The composition of the present invention has a high effect of reducing toxicity of an anti-cancer agent.

Accordingly, the present invention provides an anti-cancer adjuvant including phosphatidylcholine as an active ingredient.

The anti-cancer adjuvant refers to an agent that reduces side-effects of an anti-cancer agent or increases a therapeutic effect of the anti-cancer agent. The inventive anti-cancer adjuvant is characterized in that it includes phosphatidylcholine as an active ingredient, and has a high effect of reducing toxicity of the anti-cancer agent.

Such effects of the present invention are described in Examples.

According to one Example of the present invention, a rat, which was administered with an anti-cancer agent known to cause nephrotoxicity, cisplatin, was injected with the inventive composition. Then, it was bred and then its kidney function was measured by a blood test.

As a result, as compared to a control group that was injected intraperitoneally with cisplatin but not injected with phosphatidylcholine, a group that was injected intraperitoneally with cisplatin and then injected intraperitoneally with phosphatidylcholine showed a reduction in a blood creatinine level and a BUN (blood urea nitrogen) level (see Example 1-2).

According to another Example of the present invention, a change in the body weight of the test animal of the above described Example was measured. As a result, a control group that was injected intraperitoneally with cisplatin but not injected with phosphatidylcholine showed a reduction in body weight while a group that was injected intraperitoneally with cisplatin and then injected intraperitoneally with phosphatidylcholine showed an increase in body weight (see Example 1-3).

According to a further Example of the present invention, the kidney of the test animal of the above described Example was extracted and its tissues were observed. As a result, in a control group that was injected intraperitoneally with cisplatin but not injected with phosphatidylcholine, most epithelial cells in a proximal part and a distal part of the kidney showed a necrotic change through an inflammatory reaction by administration of cisplatin (CDDP). On the other hand, in a group that was injected intraperitoneally with cisplatin and then injected intraperitoneally with phosphatidylcholine, it was observed that epithelial cells of tubules in a proximal part and a distal part were generally well maintained (see Example 1-4).

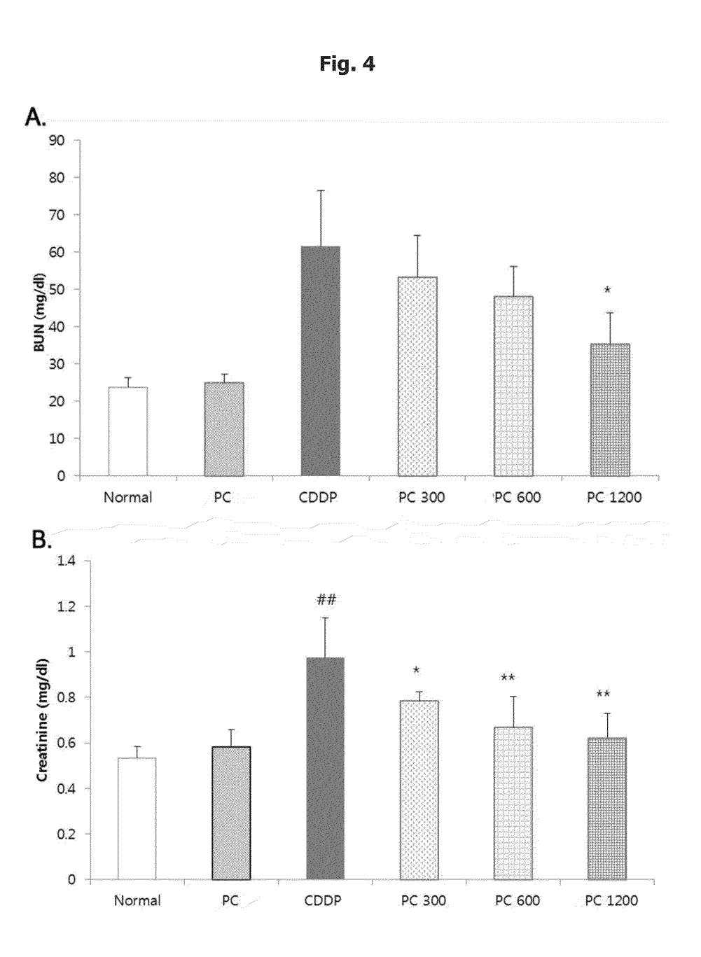

To determine the effect of oral administration, in the Example 2 phosphatidylcholine was administered orally. In case of oral administration, the group that was treated with cisplatin and then injected with phosphatidylcholine (Test group 14-16 in Table 4) showed a reduction in a blood creatinine level and a BUN (blood urea nitrogen) level than a control group that was treated with cisplatin but not injected with phosphatidylcholine (Test group 13 in Table 4). (See Example 2-2 and FIG. 4)

In addition, when phosphatidylcholine was administered orally, reducing level of oxidative stress of kidney tissue was measured.

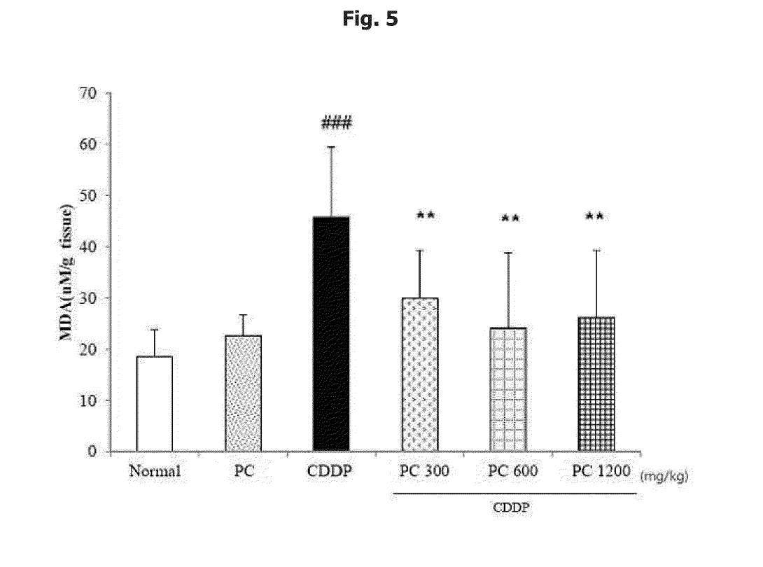

As a result of quantification of MDA (malondialdehyde) which is the is representative degradation product of lipid peroxides, the group that was treated with cisplatin and then injected with phosphatidylcholine (Test group 14-16) showed significantly low level of MDA than the group that was treated with cisplatin only (Test group 13) (See Example (1) of 2-3 and FIG. 5)

Biomembranes comprise large amount of unsaturated fatty acids. Therefore, in case that structural change of lipid molecules occurred in a broad area by lipid hyperoxidation, reduction of biomembrane fluidity, reduction of membrane potential, increase of ion permeability, leakage of contents of cell organelles occur and bring decline of cell function and cell death finally. Harmful ingredients for organism exist within lipid, oxide and their degradation product and adverse actions such as inhibition of macrophage function, inhibition of protein synthesis, inactivation of enzymes, over production of thrombin have been reported (Halliwell, B. et, al., Philos Trans R Soc B Biol Sci. December 17; 311 (1152): pp 659-671. 1985).

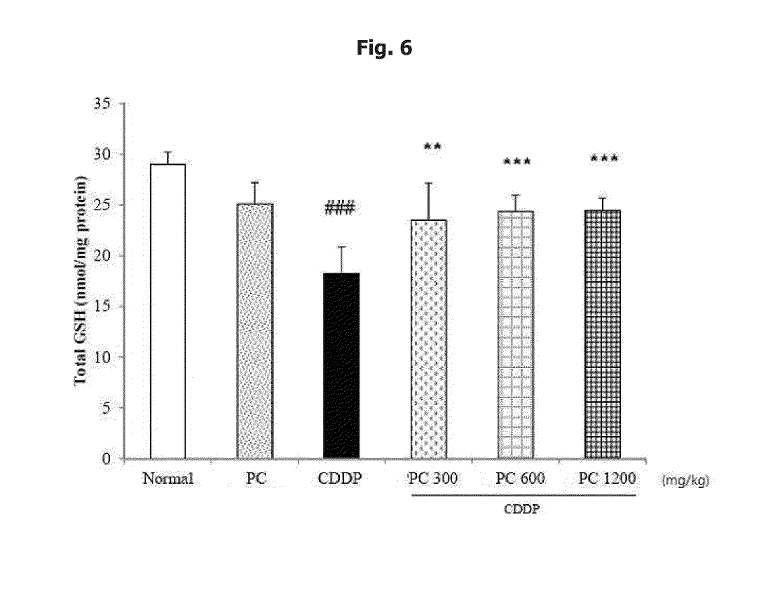

As a result of quantification of GSH (glutatione), the group that was treated with cisplatin and then injected with phosphatidylcholine (Test group 14-16) showed significantly high level of GSH concentration than the group that was treated with cisplatin only (Test group 13) (See Example (2) of 2-3 and FIG. 6).

GSH is a general term of glutathione sulfhydryl. It is a tripeptide which is constituted by binding three amino acids of glycine, glutamine and cysteine, and is synthesized in the body. It is the major endogenous antioxidant produced by the cells, participating in the neutralization of external toxic materials or endogenous free radicals, or excretion thereof.

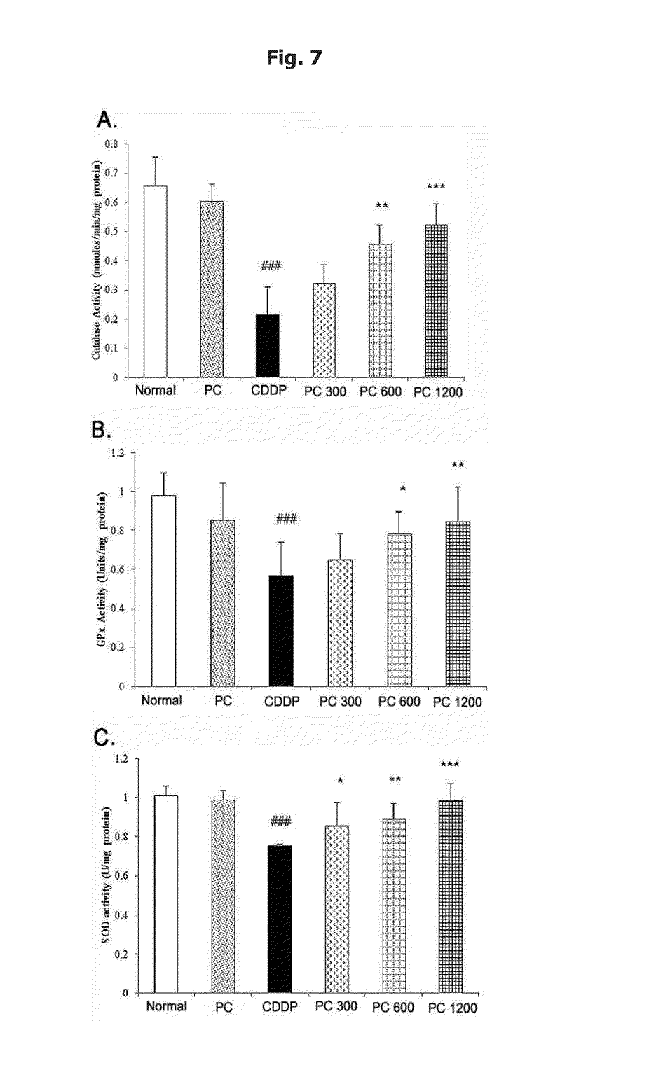

As a result of measuring activity in kidney tissue of anti-oxidant enzymes such as catalase, glutathione peroxidase (GPx), superoxide dismutase (SOD), the group that was treated with cisplatin and then injected with phosphatidylcholine (Test group 14-16) showed higher is activity level of those three anti-oxidant enzymes than the group that was treated with cisplatin only (Test group 13). (See Example (3) of 2-3 and FIG. 7).

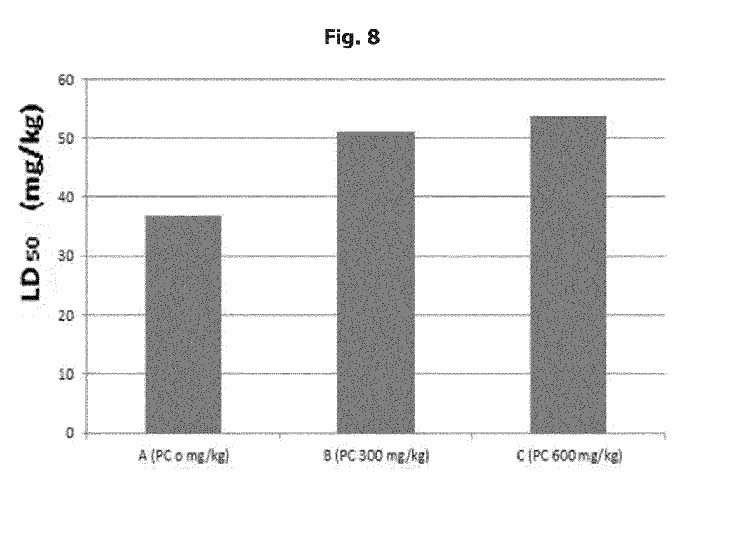

Also, we confirm that phosphatidylcholine reduces toxicity of paclitaxel in the Example 3 and FIG. 8. The present inventors found that LD₅₀ of paclitaxel increases depending on dosage of phosphatidylcholine.

A composition of the present invention comprises phosphatidylcholine having activity of reducing toxicity of an anti-cancer agent as an active ingredient and may comprise pharmaceutically acceptable carrier, diluent or excipient.

Also, administration formulation of the composition may be used as a pharmaceutically acceptable salt thereof alone or by binding or gathering with pharmaceutically active compositions.

A composition of the present invention may be used by oral formulation such as powders, granules, tablets, capsules, suspensions, emulsions, syrups, aerosols and etc., external application and sterilized injections. For a carrier, excipient and diluent, lactose, dextrose, sucrose, sorbitol, mannitol, xylitol, erythritol, maltitol, starch, acacia rubber, alginate, gelatin, calcium phosphate, calcium silicate, cellulose, methyl cellulose, crude cellulose, polyvinylpyrrolidone, water, methyl hydroxybenzoate, propyl hydroxybenzoate, talc, magnesium stearate and mineral oils can be used.

In case of formulation, a diluent such as a filler, bulking agent, binding agent, humectant, disintegrating agent, and/or a surfactant or excipient may be used. For oral administration, tablets, pills, powders, granules and capsules are comprised and one or more excipient such as starch, calcium carbonate, sucrose or lactose, gelatin can be mixed. In addition, except simple excipient, lubricant such as magnesium stearate and talk may be used. Liquid formulation for oral administration, suspensions, solution for internal use, emulsion, syrups are used and it may comprise various excipients such as humectant, sweetener, flavor and preservative, as well as simple diluent such as water and liquid paraffin.

Also, the composition of the present invention may be parenterally administered, and the parenteral administration is carried out by subcutaneous injection, intravenous injection, intramuscular injection or intra-sternal injection. For formulation of parenteral administration, phosphatidylcholine according to the present invention is prepared into a solution or a suspension liquid in mixture with a stabilizing agent or a buffer in water, and then is formulated into a unit dosage form of an ampule or a vial. There is no particular limitation in the amount of phosphatidylcholine, that is, an active ingredient of the inventive composition, but it varies according to the kind, dose, and administration period of an anti-cancer agent. It is administered preferably in an amount of about 1 to 500 times the total weight of the anti-cancer agent, more preferably of about 1 to 200 times. The inventive composition may be administered alone before or after the anti-cancer agent is administered, or may be administered as a component of an anti-cancer agent composition, in combination with the anti-cancer agent.

The effective amount may be determined preferably by considering various factors, such as health condition, body weight, disease severity, formulation of drug and administration route but it may be chosen properly by the skilled person in the art. However, for preferred effect, the composition of the present invention may be administered 0.0001 to 100 mg/kg body weight/day and preferably it may be administered 0.001 to 100 mg/kg body weight/day. Administration may be performed once a day or multiple times a day. Dosage may not be limited.

As used herein, the “subject” refers to mammals, particularly, animals comprising is human and it may be a cell, a tissue or organ originated from the animal. The subject may be patient in need of treatment.

Accordingly, the present invention provides a composition for toxicity reduction of an anti-cancer agent comprising phosphatidylcholine as an active ingredient. The composition of the present invention can reduce toxicity of an anti-cancer agent while inhibiting or minimizing various side-effects occurring by the toxicity of the anti-cancer agent during chemical therapy of cancer. Thus, it is effective as an anti-cancer adjuvant.

Hereinafter, the present invention will be described in detail with reference to following Examples. However, the following Examples are only for illustrative purposes and are not intended to limit the scope of the invention.

### Example 1

### Nephrotoxicity Reducing Effect by Cisplatin: Intra-Peritoneal Injection

<1-1> Preparation of Cisplatin and Phosphatidylcholine and Application them to a Test Animal

As cisplatin (cis-dichlorodiammineplatinum, hereinafter, referred to as ‘CDDP’), Cispatin injection from Ildong pharmaceutical was used, and phosphatidylcholine (hereinafter, referred to as ‘PC’) was prepared as described below. First, 10 kg of soybeans (scientific name: Glycine max (L.) Merill) were washed, peeled and grounded, and then at room temperature, extracted with ethanol (E.P) for 40 min. The obtained extract was filtered to remove proteins and carbohydrates, and then was vacuum evaporated at 40° C. Then, the concentrated extract was degummed and dried to remove moisture, and added with acetone. The acetone layer was separated and the residue was extracted with ethanol at 35° C. or less, for 60 min. The extract was purified with silica gel chromatography and aluminum oxide chromatography so as to provide phosphatidylcholine (essential phospholipids substance) 4 g (yield: 0.04%).

The phosphatidylcholine as prepared above was finally formulated into a micro-emulsion form, with a uniform particle size, before being administered to a Test animal according to a dose.

6-week aged male SD rats (albino S.D rat) were bought, stabilized for 1 week, and then divided into 6 groups noted in [table 1] for the test. Breeding environment conditions of 24±2° C., and 12-hour light-dark cycles were maintained, and non-antibiotic general solid feed was used. The rats used in the test had a body weight ranging from 200 g to 220 g.

TABLE 1

| No. of Group | administration material |
|---|---|
| 1 | Normal saline(0.9% NaCl) |
| 2 | PC(400 mg/kg) |
| 3 | CDDP(5 mg/kg) |
| 4 | CDDP(5 mg/kg) + PC(400 mg/kg) |
| 5 | CDDP(5 mg/kg) + PC(600 mg/kg) |
| 6 | CDDP(5 mg/kg) + PC(800 mg/kg) |

* PC: phosphatidylcholine, CDDP: cisplatin

For all groups, an agent was administered through intraperitoneal injection.

A group 1 was injected with a saline solution, groups 3, 4, 5 and 6 were injected with CDDP, and then after 1 hour, groups 2, 4, 5 and 6 were injected with PC.

They were bred while changes in their body weights were measured for 6 days. Then, after they were euthanized, their blood samples 5 cc were collected by a cardiac puncture, and their kidneys were extracted for used in a test.

<1-2> Test on a Kidney Function

The blood collected in Example <1-1> above was centrifuged at 3000 rpm for 10 min, so as to separate serum.

When nephrotoxicity occurs, levels of urea nitrogen and creatinine not filtered out due to lowering of a kidney function are increased.

The separated serum was used to measure BUN (blood urea nitrogen) and creatinine.

The measurement of BUN was carried out by using a BUN measuring kit (Young dong diagnostics) in the same manner as described below. A urea agent 0.1 ml was mixed with buffer 20 ml to prepare enzyme buffer, and the prepared enzyme buffer was added to each of 2 test tubes. To one of the test tubes, a to-be-tested serum sample 0.02 ml was added, and to the other test tube, a control reference solution [containing urea-N 60 mg/100 ml] 0.02 ml was added, followed by culturing at 37° C. for 15 min. Then, each test tube was added with chromogenic solution 2 ml, and cultured at 37° C. for 5 min again. By measuring the absorbance at 570 nm, the level of produced BUN was measured.

The measurement of creatinine was carried out by using a creatinine measuring kit (young dong diagnostics) in the same manner as described below. A to-be-tested serum sample 0.5 ml was added with tungsten solution 4 ml, and the resultant mixture was violently shaken and left for 10 min. Then, through centrifugation (1500×g) for 10 min, the supernatant was separated. Each of the separated supernatant, a creatinine standard solution and distilled water (for blank test) was added in an amount of 3 ml to a test tube. Then, each test tube was added with picrate solution 1 ml, and then 1.4N NaOH 0.5 ml, followed by sufficiently shaking. Exactly after 15 min, at 515 nm, absorbency was measured.

When CDDP was intra-peritoneally administered in a dose of 5 mg/kg, CDDP shows serious nephrotoxicity.

A creatinine level and BUN level in blood are indicators for nephrotoxicity.

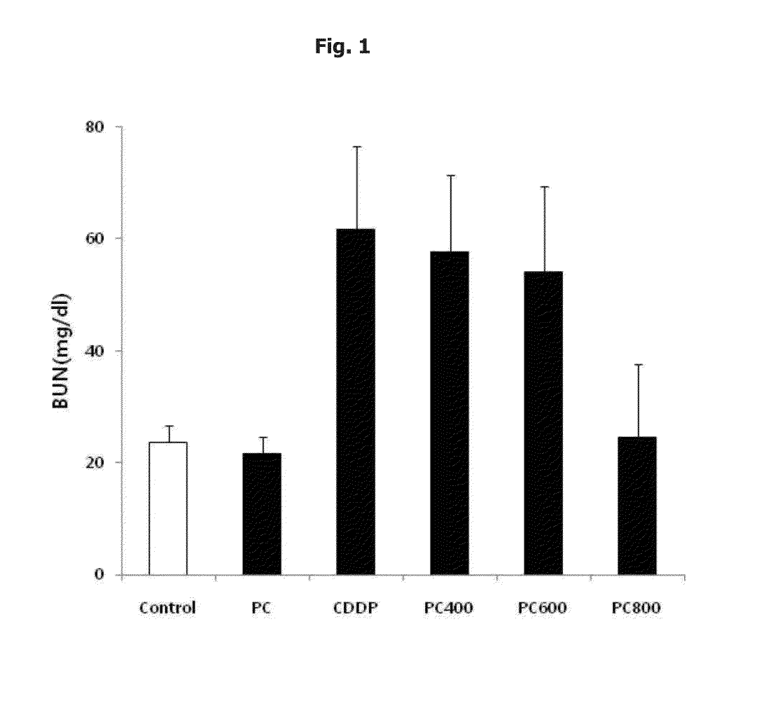

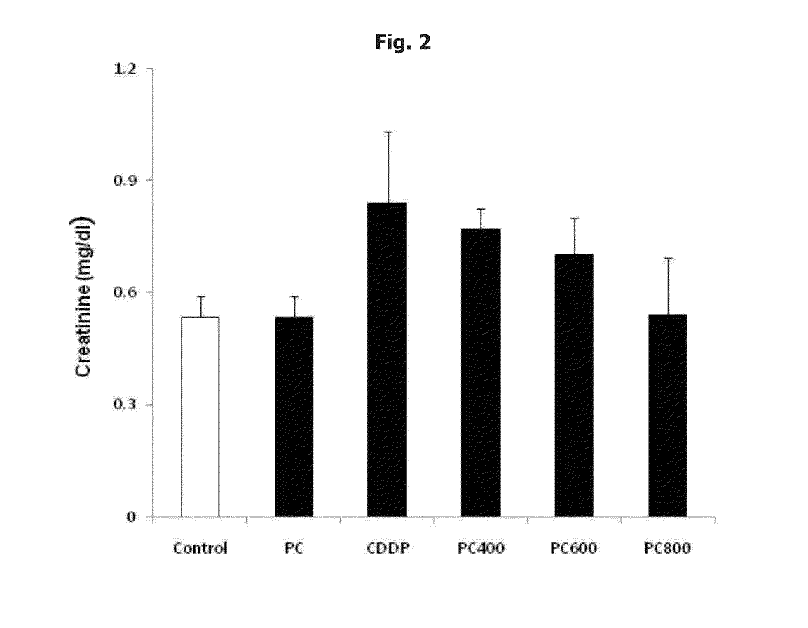

When the blood creatinine and BUN levels were measured, CDDP caused a significant increase in creatinine and BUN as shown in FIG. 1 and FIG. 2. On the other hand, when PC is administered in combination with CDDP, nephrotoxicity caused by CDDP was reduced at a concentration of 600 mg/kg or more.

<1-3> Measurement of Body Weight Rate and Lethality

During the test period, a body weight of the test group was measured.

As a result, as noted in table 2, during the test period, a body weight of a control group (group 1) was increased by 9.2% while a body weight of a CDDP-administered group (group 3) was decreased by about 7.1%. It is assumed that a decrease in the body weight was caused by CDDP toxicity.

Body weights of groups administered with CDDP in combination of PC (groups 4 and 5) were increased by about 2.3%, and 4.5%, respectively. Accordingly, it was confirmed that PC has a significant effect of inhibiting body weight reduction caused by nephrotoxicity of CDDP.

TABLE 2

| | | change of body |
|---|---|---|
| No. of Group | administration material | weight |
| 1 | Normal saline(0.9% NaCl) | +9.2% |
| 2 | PC(400 mg/kg) | +9.2% |
| 3 | CDDP(5 mg/kg) | −7.1% |
| 4 | CDDP(5 mg/kg) + PC(400 mg/kg) | +2.3% |
| 5 | CDDP(5 mg/kg) + PC(600 mg/kg) | +4.5% |

* PC: phosphatidylcholine, CDDP: cisplatin

A rats lethal dose of CDDP is 6 mg/kg. As noted in [table 3], a test group was prepared, and administered with a drug. After 6 days, lethality was calculated. An administration method, a test animal and a breeding method, etc. were the same as those in Example <1-1>.

TABLE 3

| No. of Group | administration material | lethal rate |
|---|---|---|
| 7 | Normal saline(0.9% NaCl) | 0% |
| 8 | PC(400 mg/kg) | 0% |
| 9 | CDDP(6 mg/kg) | 100% |
| 10 | CDDP(6 mg/kg) + PC(600 mg/kg) | 33.3% |

* PC: phosphatidylcholine, CDDP: cisplatin

As a result, as noted in table 3, 100% of groups not administered with CDDP (groups 7, and 8) survived, while all animals in a group administered with CDDP (group 9) died during the test period. However, in a case of a group administered with CDDP in combination with PC in a dose of 600 mg/kg (group 10), the lethality was lowered down to 33.3%.

<1-4> Observation of a Morphological Change in Kidney Tissues

A rats kidney obtained from Example <1-1> was fixed in 10% neutral formalin, sliced by microtechnique, and subjected to haematoxylin & eosin staining through a general tissue processing process. Each of the stained kidney tissues was observed by an optical microscope.

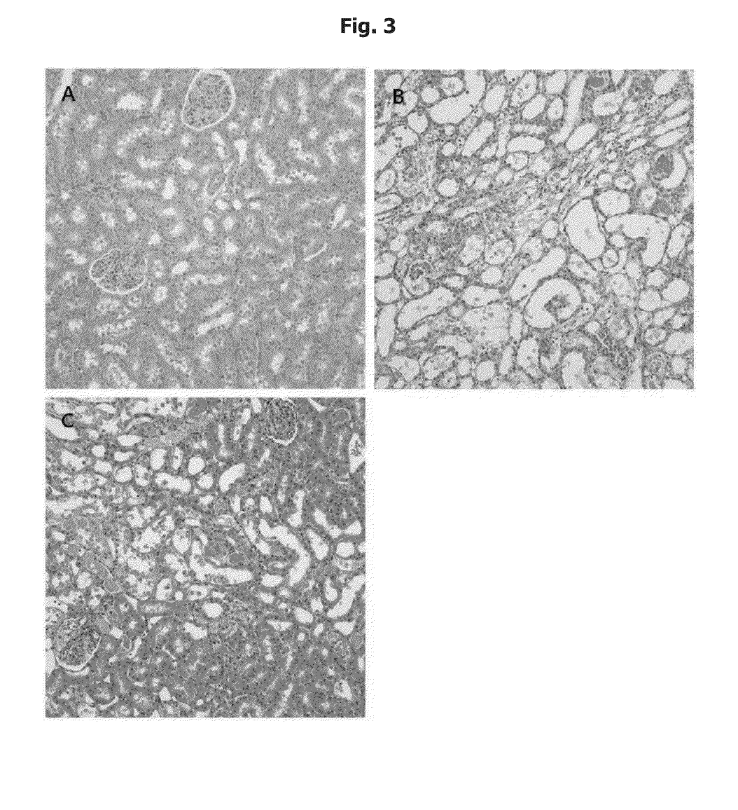

As a result, in a group (group 3) administered with only CDDP in a dose of 5 mg/kg, most epithelial cells in a proximal part and a distal part of the kidney showed a necrotic change through an inflammatory reaction (see FIG. 3-B). On the other hand, in a group (group 5) administered with CDDP in a dose of 5 mg/kg in combination with PC in a dose of 600 mg/kg, the tissues showed a damage unlike normal tissues, but it was observed that epithelial cells of tubules in a proximal part and a distal part were generally well maintained (see FIG. 3-C).

Therefore, it was confirmed that PC has an effect of significantly relieving nephrotoxicity of CDDP.

### Example 2

### Nephrotoxicity Reducing Effect by Cisplatin: Oral Administration

<2-1> Preparation of Cisplatin and Phosphatidylcholine and Application them to a Test Animal

Cisplatin and phosphatidylcholne were used as same as Example 1. But, phosphatidylcholne was suspended in 100 mg/ml of distilled water.

Thirty-six of 6-week-old adult male Wistar-Hanover rats (Nara-biotechnology, Seoul, Korea) were purchased and quarantined for 1 week and divided into 6 groups as shown in Table 4. They were maintained at 22±2° C. in 12 hour light dark cycle, and were given a normal laboratory diet (Purina. Korea) and fresh water ad libitum. Their body weight were 200˜220 g. After quarantine period, rats were fasted for 24 hours prior to injection of first phosphatidylcholine, but were allowed free access to water throughout.

TABLE 4

| No. of group | ministration material |
|---|---|
| 11 | Normal saline(0.9% NaCl) |
| 12 | PC(400 mg/kg) |
| 13 | CDDP(6 mg/kg) |
| 14 | CDDP(6 mg/kg) + PC(300 mg/kg) |
| 15 | CDDP(6 mg/kg) + PC(600 mg/kg) |
| 16 | CDDP(6 mg/kg) + PC(1200 mg/kg) |

* PC: phosphatidylcholine, CDDP: cisplatin

In all groups, saline and cisplatin were injected intraperitoneally and phosphatidylcholine was administered orally. Phosphatidylcholine was administered three times by orally at 18 hours before cisplatin injection, 30 mins after cisplatin injection, 6 hours after cisplatin injection.

Six days later, the rats were anesthetized with ether, and sacrificed. Blood samples were collected from posterior vena cava and kidney was collected.

<2-2> Analysis of Kidney Function

Blood collected in Example <2-1> was centrifuged at 4000 rpm for 10 min and serum was separated. The level of BUN (blood urea nitrogen) and creatine in serum was measured.

The level of BUN was measured by Urase-GLDH method (Laboratory reference values. Urea nitrogen (BUN). Rochester, Minn.: Mayo Foundation for Medical Education and Research; November 2010), creatine was measured by Jaffe method (Jaffe method, Lamb E et al, tiez textbook of clinical chemistry and molecular diagnosis, St. louis; elsevier saunders, 2006; 791-801) and the contents were measured Beckman Coulter AU5421 (Beckman Coulter, USA).

The measuring results were shown in FIG. 4. In FIG. 4, cisplatin brings increase of creatine and BUN and when phosphatidylcholine is administered together with cisplatin, creatine and BUN were reduced. The rate was as higher as dosage of phosphatidylcholine.

<2-3> Measurement of Oxidative Stress of Kidney Tissue

Kidney samples collected from example <2-1> were immediately removed, washed in 0.9% saline and weighed. The kidneys were the mince with scissors, were homogenized in 0.1M Tris-HCl buffer (pH 7.4). The homogenization was carried out in Teflon-glass homogenizer (Bandelin, Germany) to obtain 1:10 (w/v) dilution. The homogenate was stored at −70° C. until analysis.

Quantification of Lipid Peroxidation

Degradation products of lipid peroxidate comprise a lot of carbonyl compounds and the representative is malondialdehyde, MDA. Therefore, the level of lipid peroxidation can be measured by quantification of MDA.

For quantification of MDA, the method of Buege and Aust (1978) were used. The samples were centrifuged at 12,000 g at 4° C. for 15 min, then 0.3 ml supernatant was removed and mixed with 0.9 ml of 8% trichloroacetic acid (TCA). The sample was again centrifuged at 10,000 g at 4° C. for 5 min. A 1 ml aliquot of supernatant was added to 0.25 ml of 1% TBA, and the resulting solution was heated at 100° C. for 20 min. The tube was cooled, 2 ml of n-butanol was added, and the tube was vortexed for 90 sec. After centrifugation at 3,000 g at 4° C. for 5 min, 1 ml of the n-butanol phase was isolated and the absorbance at 532 nm was measured. The standard curve of MDA was calculated from the absorbance and the amount of MDA per weight of tissue was measured and it is shown in FIG. 5A.

MDA content was calculated with MDA standards (Buege and Aust 1978). By using molecular absorbance constant of MDA (1.56×10⁵ M⁻¹·cm⁻¹), data were shown in FIG. 5A.

In FIG. 5, MDA level of the group that was treated with cisplatin only (test group 13) is very high compared to control group 11. However, MDA level of the group administered with CDDP in combination with PC is significantly reduced compared to group 11.

Quantification of GSH (Glutathione)

The total GSH content of kidney tissue prepared in the Example <2-3> was determined using a modification of the method of Beutler et al. (Beutler, Duron et al. 1963). The method uses principle that when DTNB (5′5-dithiobis-2-nitro-benzoic acid) and GHS react, 2-nitro-5-thiobenzoic acid (yellow color) are produced. Therefore, the concentration of GSH can be determined by measuring absorbance at 412 nm.

A mitochondrial fraction was prepared by centrifugation at 600 g for 5 mins and samples were added to 5% of metaphosphoric acid (MPA) and allowed to stand for 5 mins to precipitate proteins. 1M of Phosphate buffer (pH 7.0) was added to proteins in the volumetric ratio of 1:4 for homogenization and DTNB (5′5-dithiobis-2-nitro-benzoic acid) were added to proteins in the volumetric ratio of 8:5 for color development. GSH was determined by measuring absorbance at 415 nm (shimadzu UV-1240) and absolute concentrations were calculated using a GSH standard (Sigma, USA).

In the group that was treated with cisplatin only (test group 13), the level of glutathione is significantly low compared to control group 11. However, in the group administered with CDDP in combination with PC (test group 14 to 16) the levels of glutathione is significantly increased compared to group 13 (See FIG. 6).

Measurement of activity of catalase, GPx (glutathione peroxidase), SOD (superoxide dismutase)

The activities of catalase, GPx, SOD which are anti-oxidative enzymes in kidney tissue were measured by a commercial kit.

Since catalase decomposes H₂O₂ into water and oxygen, the activity of CAT was measured through decreased absorbance by decomposition of H₂O₂. The absorbance was measured by using catalase assay kit (Sigma, CAT #100).

The activity of GPx was measured by using Glutathione peroxidase cellular activity assay kit CGP-1 (Sigma Aldrich, Cat. #CGP1). The kit used an indirect method ased upon the oxidation of glutathione (GSH) to oxidized (GSSG) catalyzed by GPx, which was then coupled with recycling of GSSG back to GSH utilizing glutathione reductase and NADPH. The decrease in NADPH absorbance measured at 340 nm, during the oxidation of NADPH to NADP is was indicative of GPx activity.

The activity of SOD is measured using the SOD assay kit (19160 SOD determination kit, Fluka/sigma Aldrich). The amount inhibiting 50% of xanthine oxidase activity per 1 mg of protein was set as 1 unit and the absorbance was measured at 450 nm.

The result is shown in FIG. 7. In the group that was treated with cisplatin only (test group 13), the levels of the anti-oxidative enzymes are significantly reduced compared to control group 11. However, in the group administered with CDDP in combination with PC (test group 14 to 16) catalase activity is significantly increased depending on the amount of PC.

(4) Statistical Analysis

The data are expressed as the means±SE. Statistical differences between means were analyzed by the Students t-test, with p<0.05 considered significant.

### Example 3

### Lethality Reducing Effect by Paclitaxel

6-week aged ICR mice (Samtako, Korea) were bought, and divided into 15 groups noted in table 5. They were stabilized for 20 hrs under conditions of 24±2° C., and 12-hour light-dark cycles while being fed with non-antibiotic general solid feed. The mice used in the test had a body weight of 25 g. After being stabilized, the mice were intra-peritoneally injected with the same phosphatidylcholine as that used in <Example 1-1>. After 4 hours, Taxol™ (BMS©, paclitaxel 6 mg/ml) was intra-peritoneally injected to the mice in which its amount was adjusted in such a manner that paclitaxel can be administered in an amount noted in table 5 below.

TABLE 5

| | Administration amount of |
|---|---|
| Group | Paclitaxel |

| | | |
|---|---|---|
| A | 1 | 10 mg/kg |
| (PC - | 2 | 30 mg/kg |
| 0 mg/Kg) | 3 | 50 mg/kg |
| | 4 | 100 mg/kg |
| | 5 | 150 mg/kg |
| B | 1 | 10 mg/kg |
| (PC - | 2 | 30 mg/kg |
| 300 mg/Kg) | 3 | 50 mg/kg |
| | 4 | 100 mg/kg |
| | 5 | 150 mg/kg |
| C | 1 | 10 mg/kg |
| (PC - | 2 | 30 mg/kg |
| 600 mg/Kg) | 3 | 50 mg/kg |
| | 4 | 100 mg/kg |
| | 5 | 150 mg/kg |

* PC: phosphatidylcholine

After 24 hours from administration of taxol, lethality of mice was measured. Based on the measured data on lethality, LD₅₀ (a paclitaxel dose at lethality of 50%) in each of A, B, and C groups was measured. The result is shown in FIG. 8.

As the administration amount of phosphatidylcholine increases, the value of LD₅₀ tends to increase. Accordingly, it can be found that phosphatidylcholine relieves toxicity of paclitaxel.

### Example of Preparation 1

### Preparation of Injection

phosphatidylcholine 100 mg

suitable amount of distilled water for injection

sodium phosphate, monobasic 2.4 mg

sodium phosphate, dibasic 2.26 mg

suitable amount of pH adjusting agent

Active ingredient is dissolved into distilled water for injection according to a well known method, and adjust pH to 7.5 and the below ingredients were dissolved in distilled water for injection. Then filled in 2 ml of ampoule, sterilized and injection were prepared.

What is claimed is:

1. A composition for toxicity reduction of an anti-cancer agent comprising phosphatidylcholine as an active ingredient.

2. The composition of claim 1, wherein the anti-cancer agent comprises cisplatin or parclitaxel.

3. The composition of claim 1, the anti-cancer agent has a toxicity selected from the group consisting of nephrotoxicity, blood toxicity, and neurotoxicity.

4. The composition of claim 1, wherein the composition comprises an amount of the phosphatidylcholine that is about 1 to 500 times an amount of the anti-cancer agent.

5. The composition of claim 1, the phosphatidylcholine is extracted from eggs or soybeans.

6. An anti-cancer adjuvant comprising phosphatidylcholine as an active ingredient.

7. Method for reducing toxicity of an anti-cancer agent comprising administering an effective amount of phosphatidylcholine to a subject in need thereof.
